(19) United States
(12) Patent Application Publication
(10) Pub. No.: US 2016/0242958 A1
(43) Pub. Date: Aug. 25, 2016

(54) THERMOTHERAPY DEVICES FOR THE EAR

(71) Applicant: Jamie Flora, Plymouth, MI (US)

(72) Inventor: Jamie Flora, Plymouth, MI (US)

(21) Appl. No.: 15/049,911

(22) Filed: Feb. 22, 2016

Related U.S. Application Data
(60) Provisional application No. 62/118,487, filed on Feb. 20, 2015.

(51) Int. Cl. A61F 7/02; A61F 7/08

(52) U.S. Cl. CPC A61F 7/02; A61F 7/08; A61F 2007/0005

(57) ABSTRACT

In a cup-shaped, double-walled enclosure that holds a hot liquid for placement over a human ear to alleviate pressure and discomfort, this invention resides in one or more structures to ensure that the enclosure remains structurally stable or does not impart thermal discomfort to a user. Such improvements or modifications include one or more of the following, alone or in combination: a thermally insulating layer of material around the outside of the enclosure; a thermally insulating layer of material around the inside of the enclosure; a post in the enclosure terminating in a distal tip indicating a preferred maximum fill level; a thermometer strip in the enclosure indicating the temperature of the liquid placed in the enclosure; and wherein the enclosure may be constructed of a thermoplastic elastomeric material that does not melt or distort when using water or other liquid thermal media at temperatures up to 280° F.

### REFERENCE TO RELATED APPLICATION

This application claims priority from U.S. Provisional Patent Application Ser. No. 62/118,487, filed Feb. 20, 2015, the entire content of which is incorporated herein by reference.

### FIELD OF THE INVENTION

This invention relates generally to thermotherapy devices and, in particular, to improvements in thermotherapy devices for the ear.

### BACKGROUND OF THE INVENTION

The Ear Ease® is a custom designed water bottle configured to hold hot water and fit over one's ear. As shown in FIG. 1, the article comprises a hard plastic base 102 defining a water reservoir and a soft, flexible cap 104 that covers the top of the unit and seals the container when pressed into place. As described in U.S. Pat. No. 5,009,228, the enclosure consists of a hollow oval channel that is a part of an additional area above, collectively identified as the “water reservoir.” The reservoir, when filled with hot water increases the temperature of the hard plastic base. The unit, once filled and securely capped is then applied to the affected area around ones ear, or other body parts, to impart a concentrated and focused heat. This localized and concentrated heat serves to accelerate blood flow in the area being treated and specifically in the case of treating the ear; it creates an increased pressure gradient in the middle ear chamber. This slight pressure serves to assist the opening of the Eustachian Tube, equalizing the pressure on both sides of the ear drum. The pent up pressure, once equalized, assists in returning the middle ear to its natural, calming and functional state.

### SUMMARY OF THE INVENTION

The present invention consists of a cup-shaped, double-walled enclosure that holds a hot liquid for placement over a human ear to alleviate pressure and discomfort, this invention resides in one or more structures to ensure that the enclosure remains structurally stable or does not impart thermal discomfort to a user. Such improvements or modifications include one or more of the following, alone or in combination: a thermally insulating layer of material around the outside of the enclosure; a thermally insulating layer of material around the inside of the enclosure; a post in the enclosure terminating in a distal tip indicating a preferred maximum fill level; a thermometer strip in the enclosure indicating the temperature of the liquid placed in the enclosure; and wherein the enclosure may be constructed of a thermoplastic elastomeric material that does not melt or distort when using water or other liquid thermal media at temperatures up to 280° F.

### BRIEF DESCRIPTION OF THE DRAWINGS

FIG. 1 is a drawing that illustrates a prior art Ear Ease® thermotherapy device;

FIG. 2 is a drawing that shown an outer thermally insulating layer around the outside of the enclosure;

FIG. 3A is a drawing that shown an outer thermally insulating layer around the inside of the enclosure;

FIG. 3B is a partial cross section of the embodiment of FIG. 3A;

FIG. 4 shows a thermal strip;

FIG. 5A shows an upright post forming a maximum fill indicator;

FIG. 5B shows a tapered maximum fill indicator; and

FIG. 5C is a top-down view of a maximum fill indicator;

### DETAILED DESCRIPTION OF THE INVENTION

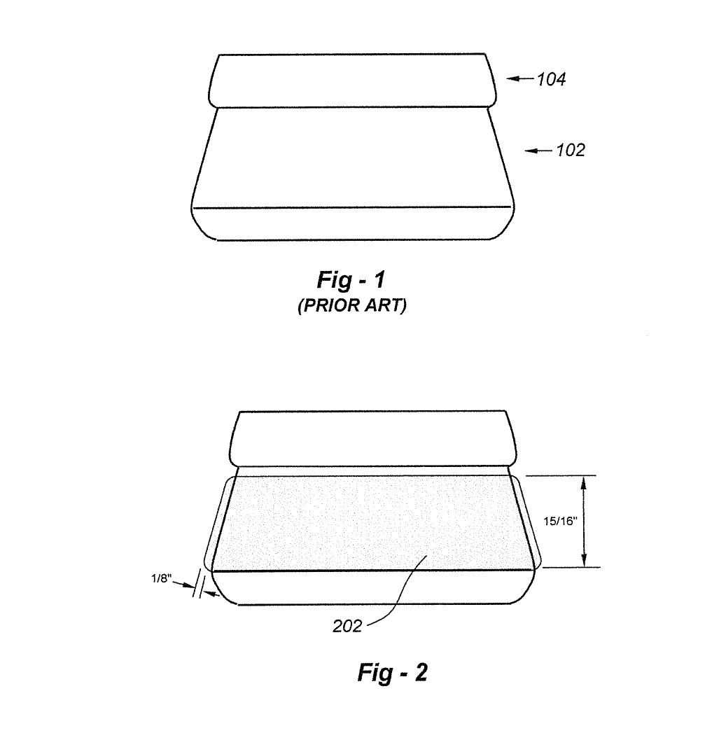

The Ear Ease® device has been marketed and sold for several years. Over that time, the inventors have noticed certain shortcomings of the device, and this disclose relates to non-obvious improvements and enhancements to the basic design of FIG. 1.

### Thermoplastic Elastomeric Materials

The existing Ear Ease comprises two components; a hard plastic molded base and a semi hard cap that when pushed onto the top of the base, creates a water tight seal. When using boiling water at 212° F., the cap has been highly susceptible to distortion and partial melting, often damaging the unit beyond further use as the seal between the cap and the base becomes compromised. This results in leakage of hot water at the junction of the cap and the base. The material in the past was limited to the technology available at the time. The ability to withstand hotter and more sustained heat was not available when the product was first introduced in 1988. Recent advances in thermoplastic elastomers have provided the ability to perform without distortion or melting when using water or other liquid thermal mediums as high as 280° F. This allows us to provide a hotter, longer lasting application of the treatment before having to refresh the hot water.

The original materials used in the production of the caps and the base of the unit were more dense and susceptible to thermal dissipation of the unit from all areas of the unit. As such, a newer material may be used, with preferred choices including Styrene Butadiene Rubber (SBR); Polybutadiene Rubber (BR); Ethylene Propylene Rubbers (EPM and EPDM); and Isoprene Rubber (IR).

The newer material is significantly softer and made of a much less dense material. The utilization of this new material, specifically for the cap, effectively acts as an insulator when holding the unit in place by hand. The large surface area of the cap reduces heat dissipation thereby extending the effective length of use before having to refill with new hot water.

The new material used in the manufacturing of both the base and the cap now allows the convenient use a Microwave oven to reheat the unit based on the higher melting point of both the cap and base materials. The walls of the unit would no longer be susceptible to distortion or collapse if heated in a microwave or filled with boiling water.

The new, more flexible material used in the manufacture of the caps makes it far more flexible, providing a superior seal and was designed to produce an audible “snap” when properly affixed to the base, reducing the potential of a partially open cap that can spill hot water on the user.

### Outer Insulating Membrane

Another issue with the existing Ear Ease device concerns elevated temperatures of the outer wall of the base component, making handling more burdensome that it could be. As such, this disclosure describes materials and techniques to insulate the base unit for better handling and to keep the water hotter for a longer period of time. FIG. 2 illustrates one such enhancement in the form of an outer insulating layer 202. To the outer wall of the base a thin 1/8″ thick rubber or rubber-like membrane can be affixed to further insulate the hand of the user. This will also provide a reduction of dissipated heat, thereby providing an extended period of use before having to recharge with hot water. The width of the barrier is on the order of 15/16″ though this width and/or the thickness of the layer may be varied in accordance with overall size and choice of material(s).

### Inner Insulating Membrane

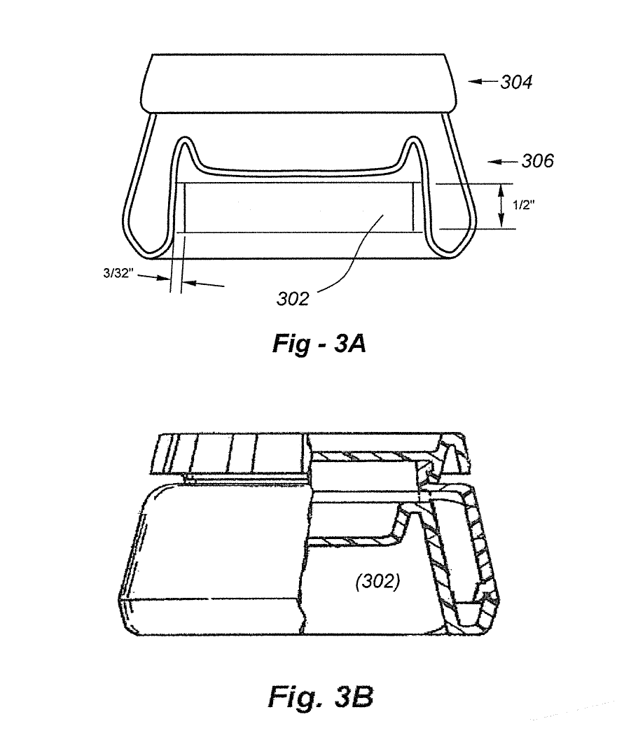

Some adults with larger ears and other individuals whose outer ears are more sensitive to high temperatures have experienced pain and discomfort when using the Ear Ease when the top of their ear and the bottom of their ear lobe make contact with the inside wall when using the Ear Ease. FIGS. 3A, B illustrate a solution to this problem. By applying a thin rubber membrane on the inside vertical walls, this insulated area will effectively address the complaints of burning of the outer ear as well as extending the effective heat life of the application. FIG. 3A is an overall perspective; FIG. 3B is a cut-away view. As seen in the drawings this inner membrane may be on the order of 3/32″ thick and ½ wide tough, as with the outer layer described above, such dimensions are variable. It should further be noted that any of the embodiments described herein may be used independently or in combination; as one example, the outer insulating layer of FIG. 2 and the inner layer of FIG. 3 may be used together.

### Thermometer Strip

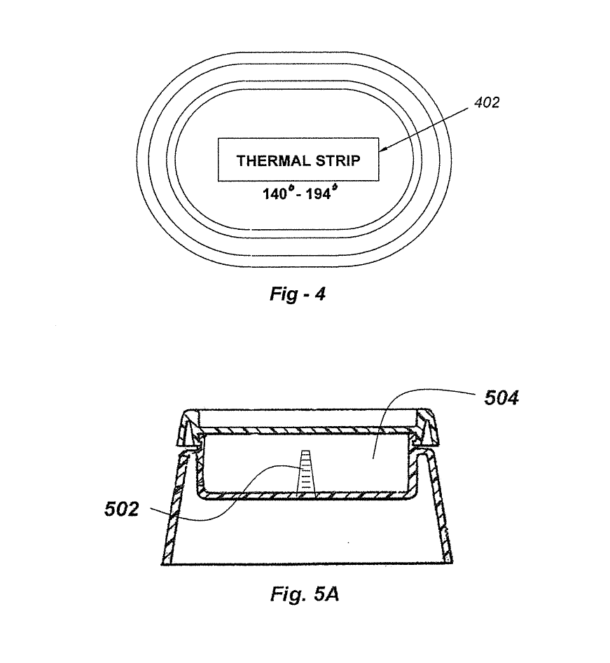

Many parents and caregivers of children deal with ear issues and there is always a concern regarding the correct temperature of the unit when applying to a newborn or an infant. This issue may be addressed by incorporating a thermometer strip on the underside of the unit to allow for instant monitoring of the temperature of the Ear Ease for better safety when using with newborns and infants. This improvement/modification is shown in FIG. 4.

### Maximum Fill Indicator

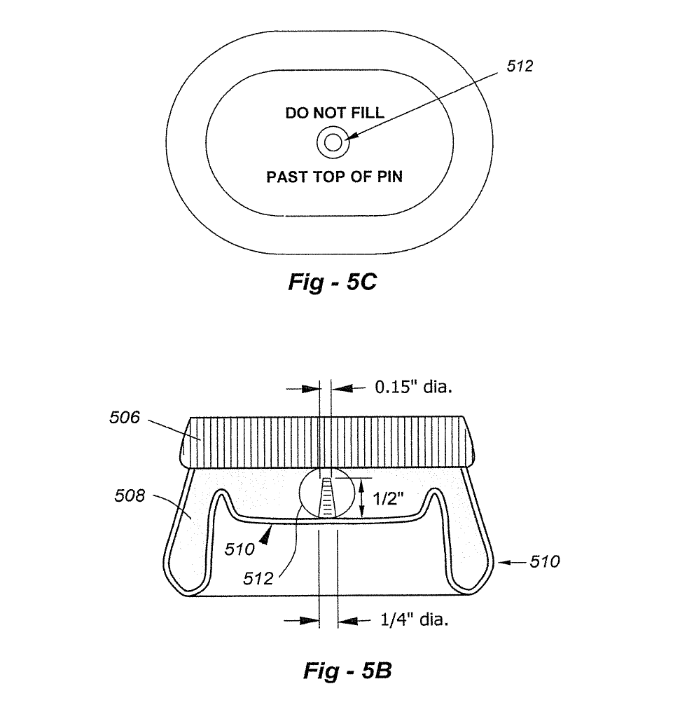

There have been cases where the end user fills the Ear Ease to the brim of the base with hot water such that when the cap is affixed, it displaces some of the hot water which spills out and may cause discomfort. FIGS. 5A-5C illustrate a maximum fill indicator to solve this problem. FIG. 5A is a side view in cross section showing this improvement. In this embodiment, the tooling of the mold is altered to provide a plastic pin 502 that will be positioned in the middle of the base unit, fully visible when the cap is removed for filling the water reservoir 504. With respect to the current unit, this pin will preferably be 0.5″ tall and 0.25″ in diameter at the base and taper to 0.15″ at the top. This will serve as a simple visual indicator of the maximum water level when the unit is being filled. Further, written instruction will be molded into the area surrounding said pin as a continual reminder that they should not overfill the unit.

FIG. 5B is a side view in partial cross section with additional details, and FIG. 5C is a top-down view of the base with the cap removed showing the pin 512 and optional instructions to avoid over-filling. The flexible cap is illustrated at 506, and the reservoir in this case is shown at 508. Surface 510 denotes the reservoir shelf, and the tapered pin is depicted at 512.

### Breather Valve

For use with a sealed version of the Ear Ease the cap can incorporate a Breather Valve so it can be used with an alternate heat medium (other than water) and can release pressure that may develop inside the Ear Ease chamber.

### Additional Embodiments

1) A “headphone” version of the invention (not shown) allows for a much larger water chamber, significantly extending the effective heat life during treatment. Each chamber would be filled from the outside of each ear cup module through a removable screw on cap. After unit is charged with hot water or another thermal medium, it would be hands free and maintain an effective heat level much longer. The unit can also incorporate a set of ear buds to provide entertainment during extended use.

2) For remote use or convenience, a custom unit fitted with a wide-mouthed reservoir that can accommodate a one time or a rechargeable thermo-pack would allow use in situations where hot water is not available.

3) An electrically heated unit may be powered by 110 AC current with a multi stepped selector that would allow for convenient home use without the trouble of emptying and refilling the unit with hot water.

1. In a cup-shaped, double-walled enclosure that holds a hot liquid for placement over a human ear to alleviate pressure and discomfort, the improvement comprising:
one or more structures to ensure that the enclosure remains structurally stable or does not impart thermal discomfort to a user.

2. The improvement of claim 1, including a thermally insulating layer of material around the outside of the enclosure.

3. The improvement of claim 1, including a thermally insulating layer of material around the inside of the enclosure.

4. The improvement of claim 1, including a maximum fill indicator in the form of a post in the enclosure, the post terminating in a distal tip indicating a preferred maximum fill level.

5. The improvement of claim 1, including a thermometer strip in the enclosure indicating the temperature of the liquid placed in the enclosure.

6. The improvement of claim 1, wherein the enclosure is constructed of a thermoplastic elastomeric material that does not melt or distort when using water or other liquid thermal media at temperatures up to 280° F.
